(19) United States
(12) Patent Application Publication
(10) Pub. No.: US 2007/0274879 A1
(43) Pub. Date: Nov. 29, 2007

(54) UV STERILIZER

(75) Inventor: Steve Millikin, St. Andrews (CA)

Correspondence Address: BLACKWELL SANDERS LLP, 720 OLIVE STREET, SUITE 2400, ST. LOUIS, MO, 63101, US

(73) Assignee: UV LIGHT SCIENCES GROUP, INC., Miramichi (CA)

(21) Appl. No.: 11/572,515

(22) PCT Filed: Jul. 25, 2005
(86) PCT No.: PCT/CA05/01152

§ 371 Date: Jan. 23, 2007

(30) Foreign Application Priority Data
Jul. 23, 2004 (US) 60590850

(51) Int. Cl. B01J 19/08

(52) U.S. Cl. 422/186.3

(57) ABSTRACT

A box-type sterilizer using C-band Ultra-Violet (UVC) radiation is based on a hollow rectangular box with a door on one end. Mounted proximate to the interior surfaces of the box are lamps that, when powered, produce UVC radiation. In operation, an object to be sterilized is placed inside the box, the door is closed and the lamps are briefly powered. Any DNA-based organisms present on the object to be sterilized are killed by the UVC radiation. Cross tubes of quartz glass, which are transparent to UVC radiation, are used to support the object to be sterilized and separate the object to be sterilized from the lamps on the bottom interior surface. Cross tubes of quartz glass are also used to separate the object to be sterilized from the lamps on the other interior surfaces.

### FIELD OF THE INVENTION

The present invention relates to sterilization using Ultra-Violet (UV) radiation and, more particularly, to a box-type UV sterilizer.

### BACKGROUND

UV sterilizers are well known for use in sterilizing all manner of objects including contact lenses, combs and safety goggles. Often only a single source of radiation is employed and, as such, there are often areas on an object to be sterilized that are shadowed from the UV radiation produced from the single source. Furthermore, the object to be sterilized is often required to rest on a support during the sterilization process. If the support is not transparent to the UV radiation, the support also contributes to shadowing the object to be sterilized from the UV radiation.

### SUMMARY

A sterilizer using C-band Ultra-Violet (UVC) radiation comprising a hollow housing with an aperture is disclosed in this specification. Mounted proximate to the interior surfaces of the housing are radiation sources that, when powered, produce UVC radiation. In operation, an object to be sterilized is placed inside the box and the radiation sources are briefly powered. Any DNA-based organisms present on the object to be sterilized are killed by the UVC radiation. Cross tubes of quartz glass, which are transparent to UVC radiation, are used to support the object to be sterilized and separate the object to be sterilized from the lamps on the bottom interior surface. Cross tubes of quartz glass may also be used to separate the object to be sterilized from the lamps on other interior surfaces.

In accordance with an aspect of the present invention there is provided a sterilizer. The sterilizer includes a housing that is opaque to Ultra-Violet radiation. The housing has an interior and an access aperture to allow ingress of an object to be sterilized and a C-band Ultra-Violet radiation transparent support structure mounted in the interior of the housing to support the object to be sterilized. The sterilizer further includes a plurality of C-band Ultra-Violet radiation sources mounted in the interior of the housing to surround the object to be sterilized on at least four sides and a plurality of C-band Ultra-Violet radiation transparent separators mounted in the interior of the housing to separate one or more of the plurality of C-band Ultra-Violet radiation sources from the object to be sterilized.

In accordance with another aspect of the present invention there is provided a sterilizer. The sterilizer includes a hollow rectangular box housing having an interior having six surfaces covered in a material that is opaque to, and reflects, Ultra-Violet radiation and a door providing a re-sealable closure for an access aperture in one of said six surfaces, said aperture allowing ingress of an object to be sterilized, a bottom C-band Ultra-Violet radiation source mounted in said interior of said housing, a C-band Ultra-Violet radiation transparent support structure mounted in said interior of said housing above said bottom C-band Ultra-Violet radiation sources, said support structure provided to support said object to be sterilized, a plurality of C-band Ultra-Violet radiation sources mounted in said interior of said housing to surround said object to be sterilized on at least four sides and a plurality of C-band Ultra-Violet radiation transparent separators mounted in said interior of said housing to separate said plurality of C-band Ultra-Violet radiation sources from said object to be sterilized.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate an exemplary embodiment of this invention:

FIG. 1 illustrates an isometric perspective view of the front, top and right sides of a UV sterilizer according to an aspect of the present invention;

FIG. 2 is a front elevation view of the UV sterilizer of FIG. 1;

FIG. 3 is a front elevation view of section C-C of the UV sterilizer of FIG. 1;

FIG. 4 is a plan view of section A-A of the UV sterilizer of FIG. 1; and

FIG. 5 is a left-side elevation view of section B-B of the UV sterilizer of FIG. 1.

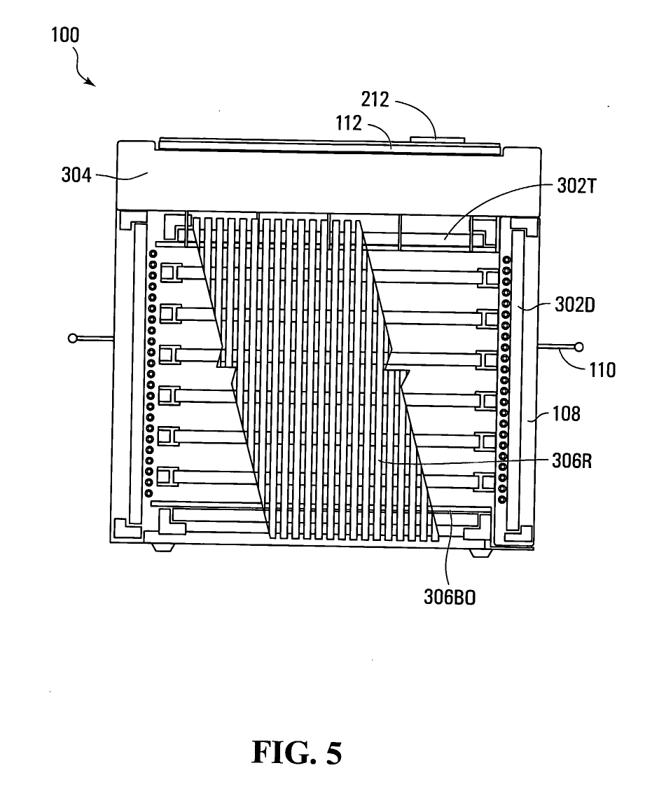

### DETAILED DESCRIPTION

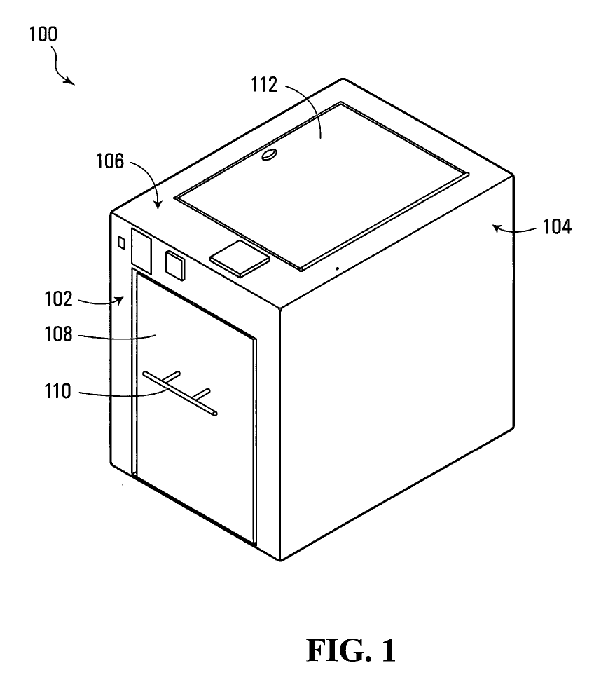

As illustrated in FIG. 1, a sterilizer 100 is formed as a box, or housing, having left and right sides, front and back sides, and top and bottom sides, where all side are opaque to UVC radiation. In the front, right perspective view of FIG. 1, the front side 102, right side 104 and top side 106 are in view. The front side 102 includes a front door 108 with a door handle 110. The top side 106 includes a top access door 112.

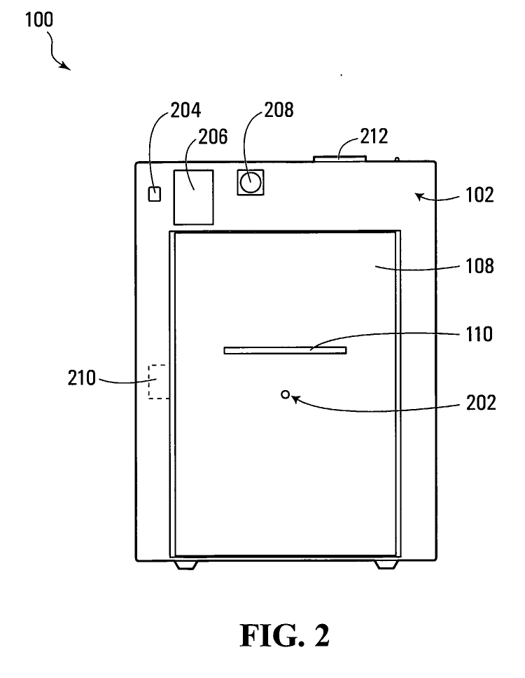

As illustrated in the elevation view of FIG. 2, the front door 108 includes a door safety switch 202 and a door lock 210. Additionally, in the portion of the front side 102 that is above the front door 108, various control elements are evident, including a master lamp power switch 204, a lamp monitor 206 and a timer 208. A fan exit port 212 is illustrated protruding slightly from the top side 106 of the sterilizer 100.

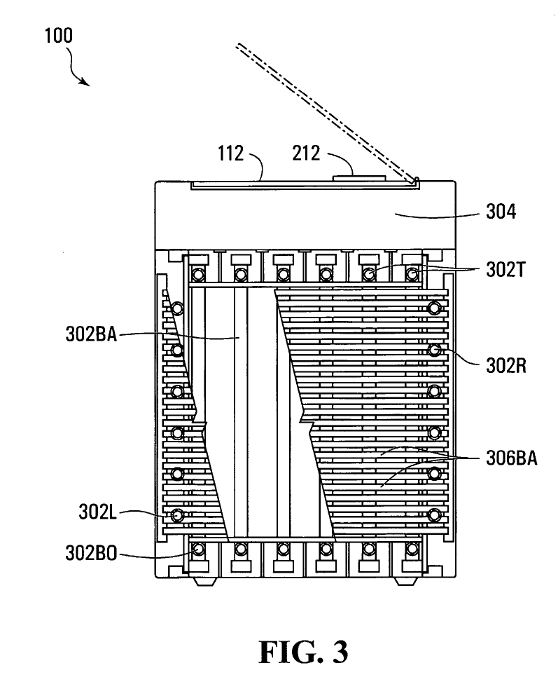

In the front elevation sectional view of FIG. 3, UVC lamps, which are individually or collectively referenced as 302, are illustrated including horizontally mounted “top” UVC lamps 302T, horizontally mounted “right” UVC lamps 302R, horizontally mounted “left” UVC lamps 302L, vertically mounted “back” UVC lamps 302BA and horizontally mounted “bottom” UVC lamps 302BO. The UVC lamps 302 may, in particular, be cold cathode UVC germicidal lamps with optional ozone lamps (not shown) or any other suitable UVC lamps. To separate the object to be sterilized from the back UVC lamps 302BA, a set of “back” quartz cross tubes 306BA (individually or collectively 306) are vertically mounted in the interior of the sterilizer 100. In one embodiment, the quartz cross tubes 306 are manufactured of pure fused hard quartz glass (also known as hard glass pure fused quartz) as this material currently allows the highest UV transparency available. Above the interior of the sterilizer 100 is an electronic compartment 304, access to which, for service of the electronic components located within the electronic compartment 304, is provided by the top access door 112.

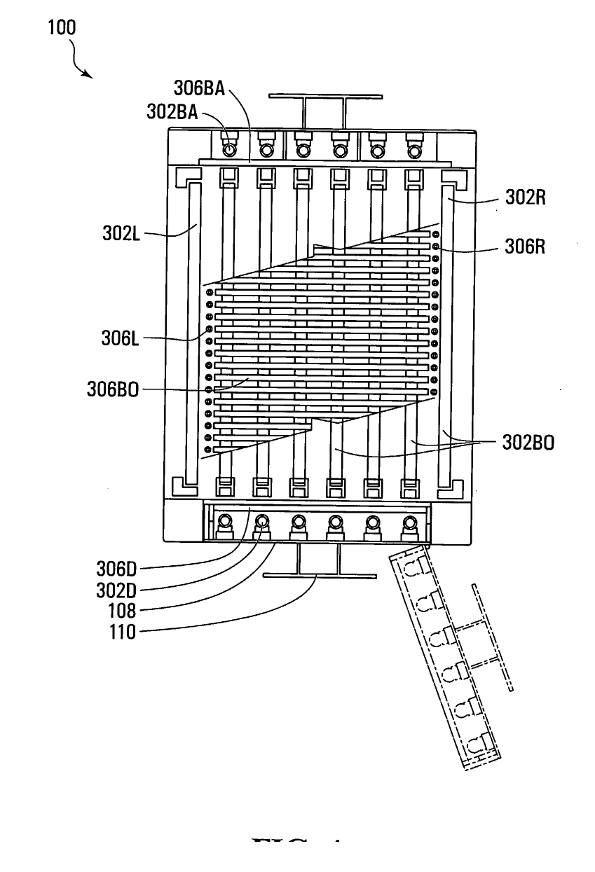

The sectional plan view of FIG. 4 allows referencing of the horizontally mounted bottom UVC lamps 302BO and vertically mounted “door” UVC lamps 302D in addition to “bottom” quartz cross tubes 306BO and “door” quartz cross tubes 306D. Additionally, a set of vertically mounted “left” quartz cross tubes 306L separate an object to be sterilized from the left UVC lamps 302L and a set of vertically mounted “right” quartz cross tubes 306R separate an object to be sterilized from the right UVC lamps 302R.

In overview, an object to be sterilized is placed in the interior of the sterilizer 100. The front door 108 is closed and the UVC lamps 302 are switched on and the object to be sterilized is bathed in UVC radiation. Due to the placement of UVC lamps 302 on many, if not all, of the interior sides of the sterilizer, few, if any, portions of the object to be sterilized are shadowed from the UVC radiation. Additionally, the object to be sterilized is supported upon the bottom quartz cross tubes 306BO, which are transparent to UVC radiation, and, therefore, do not shadow the object to be sterilized from the UVC radiation. The object to be sterilized may be further protected from contact from the array of UVC lamps 302 on each interior side of the sterilizer 100 by corresponding arrays of quartz cross tubes 306 mounted perpendicular to the UVC lamps.

In operation, a user of the sterilizer unlocks the door lock 210 of the front door 108 and opens the sterilizer 100 to expose the interior thereof. The user may then place an object to be sterilized upon the bottom quartz cross tubes 306BO in the interior, close the front door 108 and lock the door lock 210. The user may then use the timer 208 to select a desired duration of exposure and activate master lamp power switch 204 to power the UVC lamps 302. When the selected duration of exposure expires, the UVC lamps 302 may be automatically powered off. The user may then unlock the door lock 210 and open the front door 108 to remove the object, which may now be considered to be sterilized.

Sterilization may be considered the killing of all DNA-based organisms present on the object before the introduction of the object into the sterilizer 100, for example, those organisms that cause: Anthrax, Severe Acute Respiratory Syndrome (SARS), Avian Bird Flu and Creutzfeldt-Jacob Disease.

During exposure, a fan (not shown) is activated to expel heated air from the interior of the sterilizer via the fan exit port 212.

Also during exposure, the lamp monitor 206 measures and provides an indication to the user of the power output of the UVC lamps 302. It is known that, over time, the power output of the UVC lamps 302 diminishes. As the power output of the UVC lamps 302 diminishes, it is necessary to increase the duration of exposure of an object to be sterilized to radiation from the UVC lamps 302 to properly sterilize the object. Consequently, according to the indication of power output provided by the lamp monitor 206, the user may set the timer 208 appropriately for the next object to be sterilized. A person of ordinary skill in the art should be able to determine a necessary duration of radiation from the amount of radiation per unit time indicated by the lamp monitor and the desired amount of radiation to which is it is desired to expose the object to be sterilized.

Additionally, the lamp monitor 206 may be configured to indicate when the power output of the UVC lamps 302 has diminished to a level below a predetermined threshold. Such an indication may be interpreted as a sign that the UVC lamps 302 need to be changed.

In one embodiment of the present invention, if the front door 108 is opened when the sterilizer 100 is in use, i.e., when the UVC lamps 302 are powered on, the door safety switch 202 interrupts power to the UVC lamps 302. This is beneficial, since it is known that accidental exposure to UVC radiation can cause corneal burns or severe sunburn.

In one embodiment of the present invention, each of the quartz cross tubes 306 are enclosed within sleeves of Teflon™ (not shown). Advantageously, the Teflon sleeves are also transparent to UVC radiation and, in the event that one of the quartz cross tubes 306 breaks, act to contain the pieces of the broken quartz cross tube 306 for easy and tidy removal.

In one embodiment of the present invention, the interior surfaces of the sterilizer 100 may be covered with a reflective material, such as polished aluminum diamond plate, to reflect any UVC radiation that reaches an interior surface of the sterilizer 100 back toward the object to be sterilized. As will be apparent to a person of ordinary skill in the art, with these or other highly-reflective interior surfaces, the UVC lamps 302 need not necessarily be mounted proximate to all six interior surfaces of the sterilizer 100. Instead, perhaps four interior surfaces (e.g., top, bottom, left and right) would suffice.

As will be also apparent to a person of ordinary skill in the art, flat quartz glass may employed in place of the quartz cross tubes 306, in which case flat Teflon™ may be used to protect the flat quartz glass in the manner that the tube-shaped Teflon sleeves may be used to protect the quartz cross tubes 306.

Additionally, those skilled in the art will understand that the shape of the housing is not necessarily limited to that of a rectangular box. Other shapes, such as spherical and conical, may also be useful.

Other modifications will be apparent to those skilled in the art and, therefore, the invention is defined in the claims.

We claim:

1. A sterilizer comprising:
a housing that is opaque to Ultra-Violet radiation, said housing having an interior and an access aperture to allow ingress of an object to be sterilized;
a C-band Ultra-Violet radiation transparent support structure mounted in said interior of said housing to support said object to be sterilized;
a plurality of C-band Ultra-Violet radiation sources mounted in said interior of said housing to surround said object to be sterilized on at least four sides; and
a plurality of C-band Ultra-Violet radiation transparent separators mounted in said interior of said housing to separate one or more of said plurality of C-band Ultra-Violet radiation sources from said object to be sterilized.

2. The sterilizer of claim 1 wherein surfaces of said interior of said housing are covered with a reflective material.

3. The sterilizer of claim 2 wherein said reflective material is polished aluminum diamond plate.

4. The sterilizer of claim 1 wherein said support structure comprises a shelf of quartz tubes.

5. The sterilizer of claim 4 wherein said quartz tubes are manufactured of pure fused hard quartz glass.

6. The sterilizer of claim 4 wherein said quartz tubes are surrounded by a flexible, C-band Ultra-Violet radiation transparent coating.

7. The sterilizer of claim 6 wherein said C-band Ultra-Violet radiation transparent coating is Teflon™.

8. The sterilizer of claim 1 wherein said support structure comprises a shelf of quartz plate.

9. The sterilizer of claim 8 wherein said quartz plate is manufactured of pure fused hard quartz glass.

10. The sterilizer of claim 8 wherein said quartz plate is coated on a top side and a bottom side by a flexible, C-band Ultra-Violet radiation transparent coating.

11. The sterilizer of claim 10 wherein said C-band Ultra-Violet radiation transparent coating is Teflon™.

12. The sterilizer of claim 1 wherein said separators are a plurality of quartz tubes.

13. The sterilizer of claim 1 including a monitor adapted to determine a measured output of radiation from said plurality of C-band Ultra-Violet radiation sources and to provide an indication of said measured output external to said housing.

14. The sterilizer of claim 13 including a monitor providing said indication of said measured output relative to a threshold.

15. The sterilizer of claim 1 further comprising a timer to limit a duration of operation of said C-band Ultra-Violet radiation sources.

16. The sterilizer of claim 1 further comprising:
an exhaust aperture providing communication between said interior of said housing and an exterior of said housing; and
a fan to assist expulsion of air from said interior of said housing.

17. The sterilizer of claim 1 including a door to prevent egress of radiation produced by said plurality of Ultra-Violet radiation sources through said aperture.

18. The sterilizer of claim 17 including a further C-band Ultra-Violet radiation source mounted on an interior of said door.

19. The sterilizer of claim 18 including a plurality of C-band Ultra-Violet radiation transparent separators mounted on said interior of said door between said further C-band Ultra-Violet radiation source and said object to be sterilized.

20. A sterilizer comprising:
a hollow rectangular box housing having:
an interior having six surfaces covered in a material that is opaque to, and reflects, Ultra-Violet radiation; and
a door providing a re-sealable closure for an access aperture in one of said six surfaces, said aperture allowing ingress of an object to be sterilized;
a bottom C-band Ultra-Violet radiation source mounted in said interior of said housing;
a C-band Ultra-Violet radiation transparent support structure mounted in said interior of said housing above said bottom C-band Ultra-Violet radiation sources, said support structure provided to support said object to be sterilized;
a plurality of C-band Ultra-Violet radiation sources mounted in said interior of said housing to surround said object to be sterilized on at least four sides; and
a plurality of C-band Ultra-Violet radiation transparent separators mounted in said interior of said housing to separate said plurality of C-band Ultra-Violet radiation sources from said object to be sterilized.
